(19) United States
(12) Patent Application Publication
(10) Pub. No.: US 2020/0197011 A1
(43) Pub. Date: Jun. 25, 2020

(54) LINE-COMPRESSION PATCH

(71) Applicant: TERUMO KABUSHIKI KAISHA, Tokyo (JP)

(72) Inventors: Masakatsu KAWAURA, Sunnyvale, CA (US); Shuji UEMURA, San Francisco, CA (US); David R. BATTEN, San Francisco, CA (US)

(73) Assignee: TERUMO KABUSHIKI KAISHA, Tokyo (JP)

(21) Appl. No.: 16/802,772

(22) Filed: Feb. 27, 2020

Related U.S. Application Data
(63) Continuation of application No. PCT/JP2018/030702, filed on Aug. 20, 2018.
(60) Provisional application No. 62/553,284, filed on Sep. 1, 2017.

(51) Int. Cl. A61B 17/12

(52) U.S. Cl. CPC A61B 17/12; A61B 2017/00867

(57) ABSTRACT

A line-compression patch and method are disclosed configured to achieve hemostasis in a living body. The line-compression patch includes a line-compression feature extruding out towards a patient contact surface; a base providing the support to the line-compression feature; and a patch feature that provides stability to the line-compression patch by adhering to the patient contact surface.

### CROSS-REFERENCES TO RELATED APPLICATIONS

This application is a continuation of International Application No. PCT/JP2018/030702 filed on Aug. 20, 2018, which claims priority to U.S. Provisional Patent Application No. 62/553,284, filed on Sep. 1, 2017, the entire content of both of which is incorporated herein by reference.

### TECHNICAL FIELD

The present disclosure generally relates to a medical device, more particularly a line-compression patch and methods for achieving hemostasis with a line-compression patch, and more particularly, a line-compression patch, which are configured, for example, to achieve hemostasis at femoral vein access site after cardiac ablation, left atrial appendage closure, or mitral valve repair by percutaneous catheter procedure.

### BACKGROUND DISCUSSION

Known methods of performing hemostasis can include manual compression, figure of eight suturing, and arterial closure device or hemostasis tools. Manual compression is relatively easy; however, one typically must hold compression for 10 to 30 min, requires long bed rest (4 to 6 hours) which causes back pain, and more likely to bleed again. The figure-of-eight suturing is faster (about 1 min); however, it can require training and has potential for perforation (deep puncture), bleeding (shallow suture path), and embolization (too tight). The arterial closure devices have strong hemostatic capability; however, they often have high cost, may require more than one device for a large-bore site, and can potentially injure venous wall.

It would be desirable to have a line-compression patch, that can achieve hemostasis which allows the patient to ambulate relatively quickly, in a manner that is more consistent and reliable (thus safer) than the figure-of-eight suturing, and cheaper and safer than the arterial closure devices.

### SUMMARY

A line-compression patch configured to achieve hemostasis at femoral vein access site in a living body is disclosed, the line-compression patch comprising: a line-compression feature that extends towards a patient contact surface and applies concentrated pressure to seal off the bleeding wound channel(s); a base in which a line-compression feature attaches and provides structural support to the line-compression feature; a patch feature that attaches to the base and provide stability to the line-compression patch by adhering to the patient contact surface.

A line-compression patch configured to achieve hemostasis in a living body is disclosed, the line-compression patch comprising: a separate adhesion patch that consists of a patch, a skin adhesion feature, a skin adhesion cover, and a patch assembly adhesion feature, and wherein the skin adhesion feature is on a front side of the patch that meets a patient's skin, and is covered by the skin adhesion cover until the separate adhesion patch is ready to be adhered to the patient, and on a back side of the separate adhesion patch, the patch assembly adhesion feature is attached to the patch; and an adhesion feature having a line-compression feature extruding out towards a patient contact surface, and wherein the adhesion feature is configured to connect to the patch assembly adhesion feature.

A method is disclosed of achieving hemostasis at femoral vein access site in a living body, the method comprising: placing a line-compression patch on the skin over the bleeding wound channel(s) and adhering the patch feature to the skin to keep the line compression patch in place; the line-compression feature that extends towards the patient contact surface which applies concentrated pressure to seal off the wound channel(s); the base that provides structural support for the line compression feature; the patch feature that provides stability to the line-compression patch by adhering to the patient contact surface.

### BRIEF DESCRIPTION OF THE DRAWINGS

FIG. 1 is a perspective view of a line-compression patch assembly (shown with a curved wire) in accordance with an exemplary embodiment.

FIG. 2 is an illustration of the placement of a line-compression patch assembly on an access site in accordance with an exemplary embodiment.

FIG. 3 is a perspective view of a line-compression patch assembly with a rectangular-shaped rod in accordance with another exemplary embodiment.

FIG. 4A is a perspective view of a line-compression patch assembly with a thin plate configuration, wherein FIG. 4A is semicircular profile, in accordance with another exemplary embodiment.

FIG. 4B is a perspective view of a line-compression patch assembly with a thin plate configuration, wherein FIG. 4B is rectangular profile, in accordance with another exemplary embodiment.

FIGS. 4C-4E are perspective views of the line-compression patch assembly with a thin rectangular plate with radius on each corner in accordance with an exemplary embodiment.

FIG. 4F is a schematic view of the line-compression patch with a thin plate configuration with a uniform thickness in accordance with an exemplary embodiment.

FIG. 4G is a schematic view of the line-compression patch with a thin plate configuration with a tapered thickness in accordance with an exemplary embodiment.

FIG. 5A is a perspective view of a line-compression patch assembly with multiple parallel curved wires, wherein FIG. 5A is with two parallel curved wires, in accordance with another exemplary embodiment.

FIG. 5B is a perspective view of a line-compression patch assembly with multiple parallel curved wires, wherein FIG. 5B is with five parallel curved wires, in accordance with another exemplary embodiment.

FIGS. 5C-5E are perspective views of a line compression assembly with two or more opposing-angled curved wires in accordance with another exemplary embodiment.

FIG. 6A is a perspective view of a line-compression patch assembly with varying edge curvature in accordance with another exemplary embodiment.

FIGS. 6B-6D are a top view, a front view, and a side view, respectively of the line-compression patch assembly with varying edge curvature as shown in FIG. 6A.

FIG. 6E is a perspective view of a line-compression patch assembly with varying edge curvature in accordance with another exemplary embodiment.

FIG. 6F is a perspective view of a line-compression patch assembly with varying edge curvature in accordance with another exemplary embodiment.

FIGS. 6G-6I are perspective views of a line-compression patch assembly with varying edge curvature in accordance with another exemplary embodiment.

FIG. 7A is a perspective view of a line-compression patch assembly with a relief feature on the base in accordance with another exemplary embodiment.

FIG. 7B is a perspective view of a line-compression patch assembly with a relief feature on the base in accordance with another exemplary embodiment.

FIG. 8A is a perspective view, in accordance with another exemplary embodiment.

FIG. 8B is a schematic view, in accordance with another exemplary embodiment.

FIG. 8C is an illustration of a line-compression patch assembly with an adjustment feature, in accordance with another exemplary embodiment.

FIG. 9A is a perspective view, in accordance with another exemplary embodiment.

FIGS. 9B-9D are detailed views, in accordance with another exemplary embodiment.

FIG. 9E is an illustration of a line-compression patch assembly with another adjustment feature, in accordance with another exemplary embodiment.

FIGS. 10A and 10B are perspective views of a line-compression patch assembly with a separate adhesion patch in accordance with another exemplary embodiment.

FIGS. 10C and 10D are perspective views of a line-compression patch assembly with a separate adhesion patch in accordance with another exemplary embodiment.

FIG. 10E is an illustration of a line-compression patch assembly with a separate adhesion patch in accordance with another exemplary embodiment.

FIG. 11A shows a line-compression patch assembly with a pivoting adjustment feature in accordance with an exemplary embodiment.

FIG. 11B shows a line-compression patch assembly with a pivoting adjustment feature in accordance with an exemplary embodiment.

### DETAILED DESCRIPTION

Hereinafter, embodiments of the present disclosure will be described with reference to the drawings. In order to facilitate description, dimensional ratios in the drawings are exaggerated, and thus are different from actual ratios in some cases.

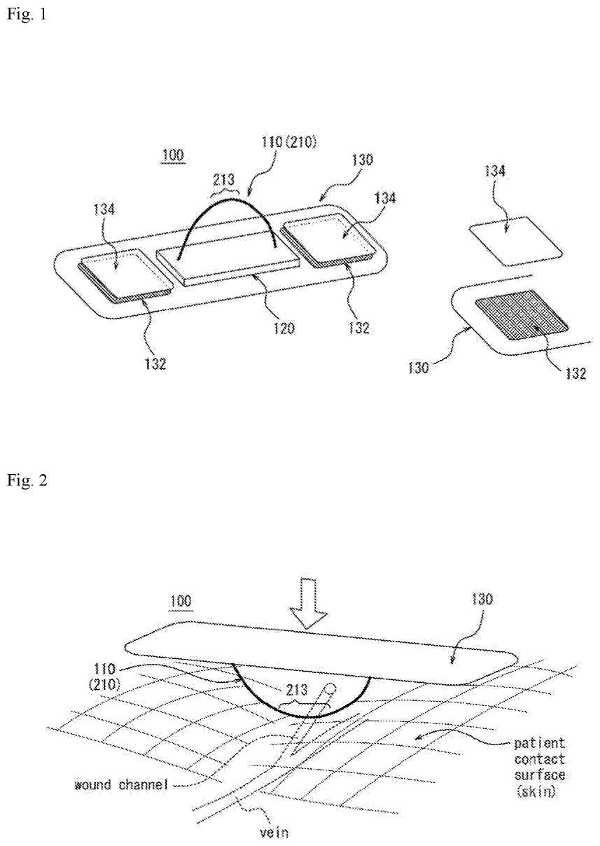

FIG. 1 is a perspective view of a line-compression patch assembly 100 with an exemplary embodiment. As shown in FIG. 1, the line-compression patch assembly 100 includes the line-compression feature 110, the base 120, the patch feature 130, the adhesion feature 132, and the adhesion cover 134. In accordance with an exemplary embodiment, the line-compression feature 110 can be pressed against the target area of the patient by placing the patch feature 130 flat against the patient's skin. The base 120 provides vertical stability to the line-compression feature 110 with respect to the contact surface such that it does not tilt under compression. The adhesion feature 132 is on the surface of the patch feature 130 that meets the patient's skin, and is covered by the adhesion cover 134 until the patch is ready to be adhered to the patient. The adhesion feature 132 and the matching adhesion cover 134 may be separate and be on either side of the line compression feature 110 as shown in FIG. 1, or they may each be a single piece that is connected along the length of the patch feature 130. Once hemostasis is achieved, the line-compression patch assembly 100 is removed. In accordance with an exemplary embodiment, the line-compression feature 110 is of a height that does not reach the blood vessel of the patient. In accordance with an exemplary embodiment, the line-compression feature 110 can be directly connected to the patch feature 130 without the base 120. In accordance with an exemplary embodiment, when connecting the line-compression feature 110 directly to the patch feature 130, at least a part of the patch feature 130 can be made of a harder material, for example, than other parts of the patch feature 130.

FIG. 2 is an illustration of the preferred orientation of the line-compression feature 110 on the contact surface plane wherein the line-compression feature 110 is perpendicular to the flow of the vein or the wound channel(s). The peak 213 or the midpoint of the curved wire is in alignment with the patient's vein and the wound channel(s).

The line-compression feature 110 described in FIGS. 1 and 2 may vary in its configuration and numbers. In FIGS. 1 and 2, the line-compression feature is depicted as a curved wire 210. In accordance with an exemplary embodiment, the wire can be, for example, approximately 0.5 mm to 2.5 mm in diameter, 0.5 mm×0.5 mm to 2.5 mm×2.5 mm square cross section, or 0.5 mm to 2.5 mm by 0.5 mm to 2.5 mm rectangular cross section; the distance between the ends of the curved wire 210 on the base 120 can be, for example, approximately 10 mm to 50 mm, the distance from the base 120 to the peak of the arc 213 can be, for example, approximately 3 mm to 20 mm.

The curved wire 210 of the line-compression patch assembly 100 may be made of, for example, stainless steel, nickel titanium (NiTi) alloy, Elgiloy, polypropylene, polyethylene, and polyethylene terephthalate (PET). The patient contact area of the curved wire 210 may have a different texture, for example, tacky, roughened, or abraded to prevent the curved wire 210 from slipping or sliding. A piece of tubing with different texture and material characteristics such as polyurethane, polyether block amide (for example, PEBAX), polyvinyl chloride (PVC), Nitryl, silicone, or braided tubing using any combination of the above materials may be placed over the wire, which may prevent the curved wire 210 from slipping or sliding and also relieve potential pain to the patient.

FIG. 2 also illustrates the peak of the arc 213 aligning with the vein and the wound channel(s) when applied to the patient, wherein, the wire may provide spring force to absorb the compressive force to conform and compensate for the patient's movement while applied to the patient.

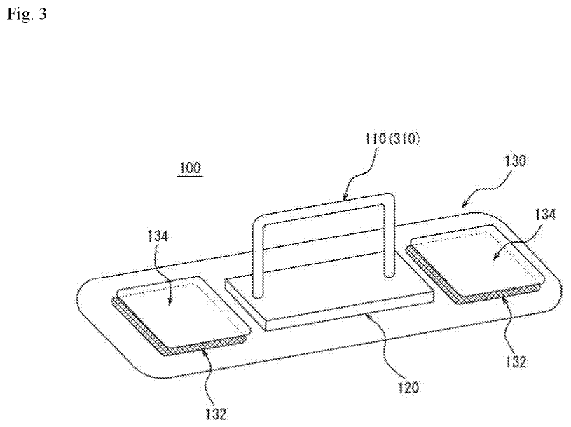

FIG. 3 is a perspective view of the line-compression feature 110 with a rectangular-shaped rod 310. In accordance with an exemplary embodiment, the rod can be, for example, approximately 1 mm to 3.5 mm in diameter, 1 mm×1 mm to 3.5 mm×3.5 mm square cross section, or 1 mm to 3.5 mm by 1 mm to 3.5 mm rectangular cross section; the distance between the ends of the rectangular-shaped rod 310 on the base 120 can be, for example, approximately 10 mm to 50 mm, the height from the base 120 can be, for example, approximately 3 mm to 20 mm.

The rectangular-shaped rod 310 of the line-compression patch assembly 100 may be made of, for example, stainless steel, nickel titanium (NiTi) alloy, Elgiloy, polycarbonate, acrylonitrile butadiene styrene (ABS), PET, and acrylic. The patient contact area of the rectangular-shaped rod 310 may have a different texture, for example, tacky, roughened, or abraded to prevent the rectangular-shaped rod 310 from slipping or sliding. A piece of tubing with different texture and material characteristics such as polyurethane, PEBAX, PVC, nitryl, silicone, or braided tubing using any combination of the above materials may be placed over the wire, which may prevent the rectangular-shaped rod 310 from slipping or sliding and also relieve potential pain to the patient.

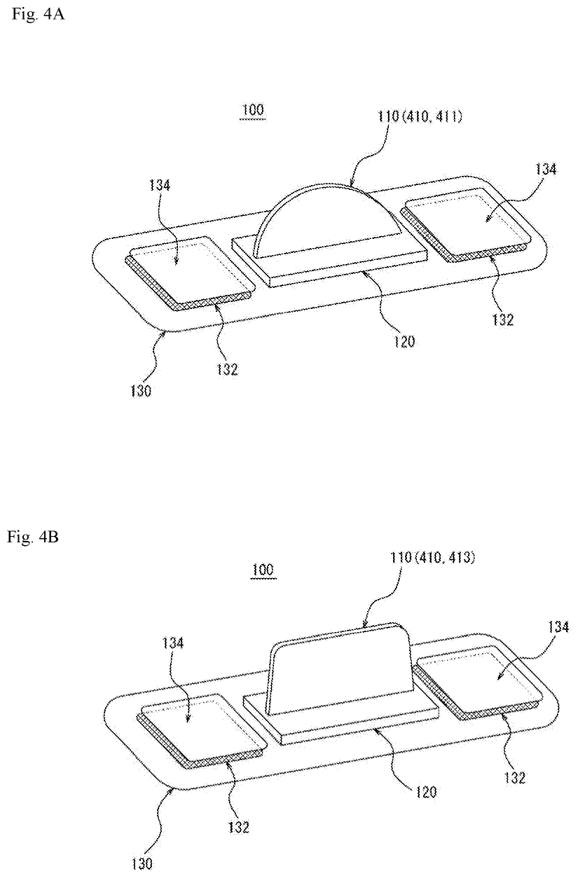

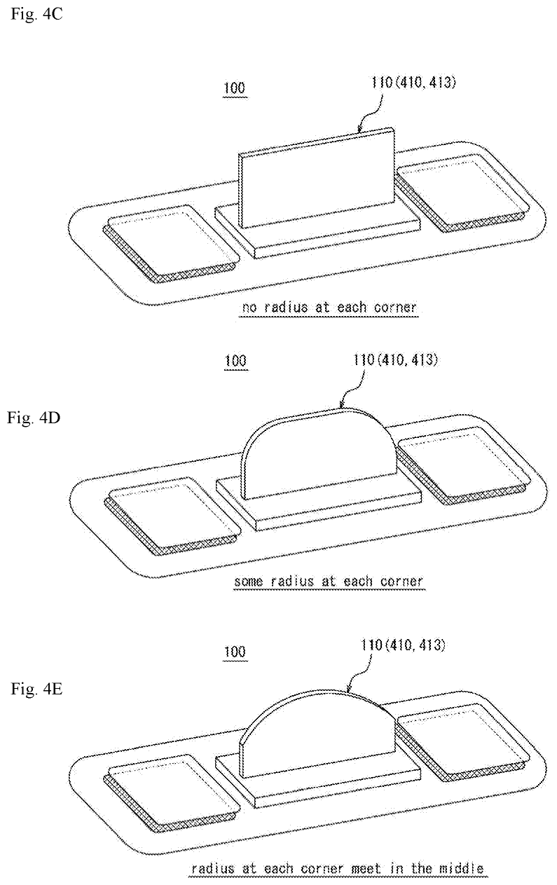

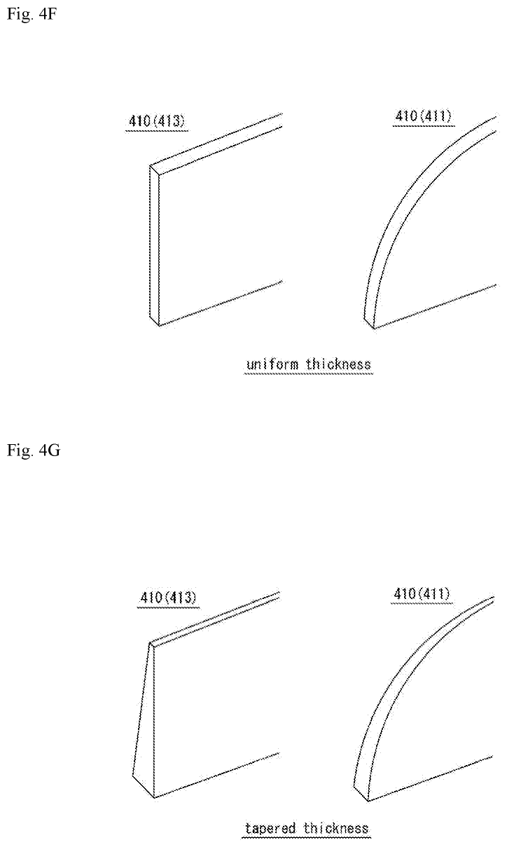

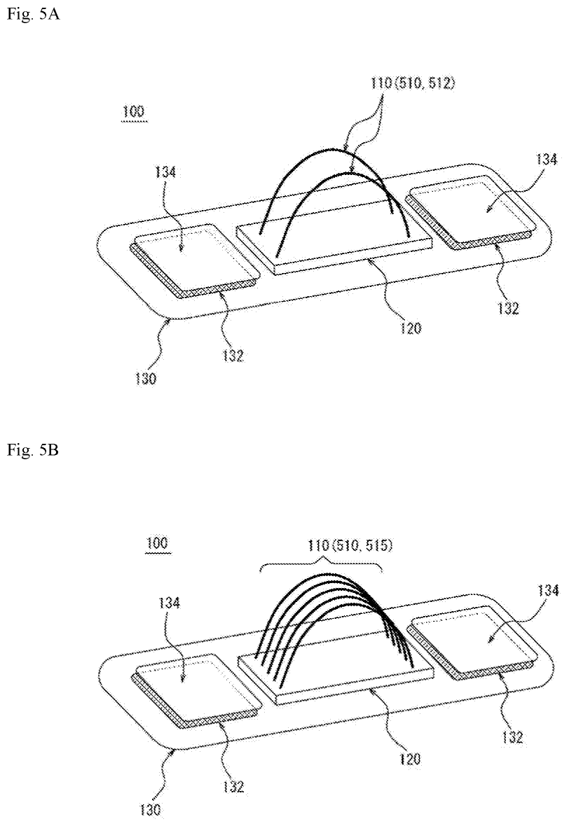

FIGS. 4A and 4B are perspective views of the line-compression feature 110 with a thin plate configuration 410. The profile of the thin plate can be semicircular 411 (FIG. 4A), or rectangular 413 (FIG. 4B). In accordance with an exemplary embodiment, the bottom of a thin plate configuration 410 can be, for example, approximately 10 mm to 50 mm in length and the height can be approximately 3 mm to 20 mm.

The thin plate 410 of the line-compression patch assembly 100 may be made of, for example, polycarbonate, ABS, polyethylene, polyurethane, PET, acrylic, silicone, PEEK, stainless steel, nickel titanium (NiTi) alloy, or Elgiloy. The patient contact area of the thin plate 410 may have a different texture, for example, tacky, roughened, or abraded to prevent the thin plate 410 from slipping or sliding.

Each corner of the rectangular profile 413 can have a uniform or nonuniform radius that can be, for example, between zero and a half of the profile length where two radii from both corners meet in the middle (FIGS. 4C-4E). The angle of each edge of the rectangular profile 413 with respect to the base 120 can be, for example, approximately 90° to 150°.

FIG. 4F is a schematic view of the thin plate configuration 410 with a uniform thickness, wherein, the thickness can be, for example, approximately 0.5 mm to 2.5 mm.

FIG. 4G is a schematic view of the thin plate configuration 410 with a tapered thickness, wherein, the thickness can be such that the tapered portion which is furthest away from the base 120 is less than the thickness at the base 120 that can be, for example, approximately 0.5 mm to 2.5 mm.

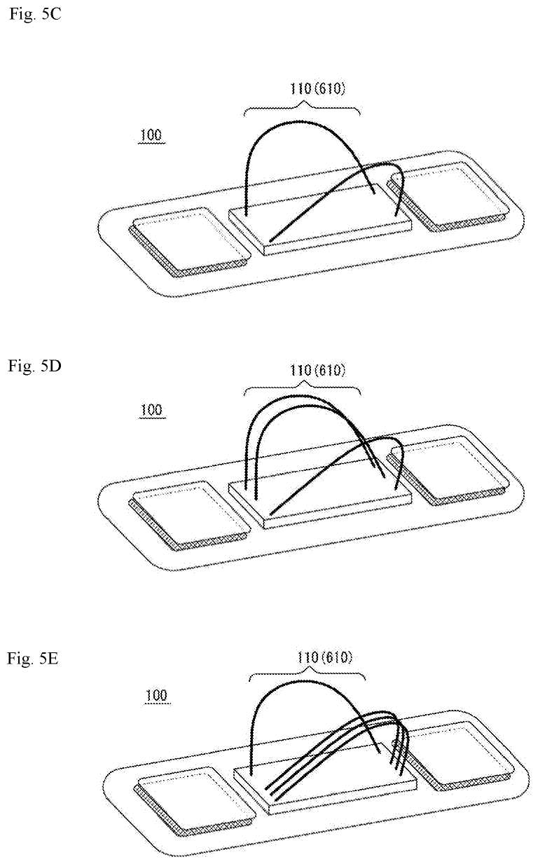

All the above configurations (210, 310, and 410) of the line-compression feature 110 may vary in number from one to five 510. FIGS. 5A and 5B are perspective views of a line-compression assembly with two parallel curved wires 512 and five parallel curved wires 515, respectively. Any of the line-compression features 110 can have the same or different height from one another, for example, approximately 3 mm to 20 mm, according to patient's anatomy, size and number of the sheaths; the distance between any adjacent line-compression features 110 can be, for example, approximately 3 mm to 30 mm.

All the above configurations (210, 310, 410, and 510) of the line-compression feature 110 may vary in the angle formed between the line-compression feature 110 and the base 120. The angle formed between the line-compression feature 110 and the base 120 can be, for example, approximately 30° to 90°.

FIGS. 5C-5E are perspective views of a line-compression feature 110 with two or more opposing-angled curved wires 610. In these configurations, curved wire(s) oriented in one direction provides the line-compression to the wound channel while the curved wire(s) in opposing direction provides additional support and stability. The number of curved wire oriented in either direction may vary from one to five. The angle formed between the line compression feature 110 and the base 120 can be, for example, approximately 30° to 90°. The opposing-angled curved wires 610 may be allowed to flex or bend from the base 120 and the wire may provide spring force to absorb the compressive force to conform and compensate for the patient's movement while applied to the patient.

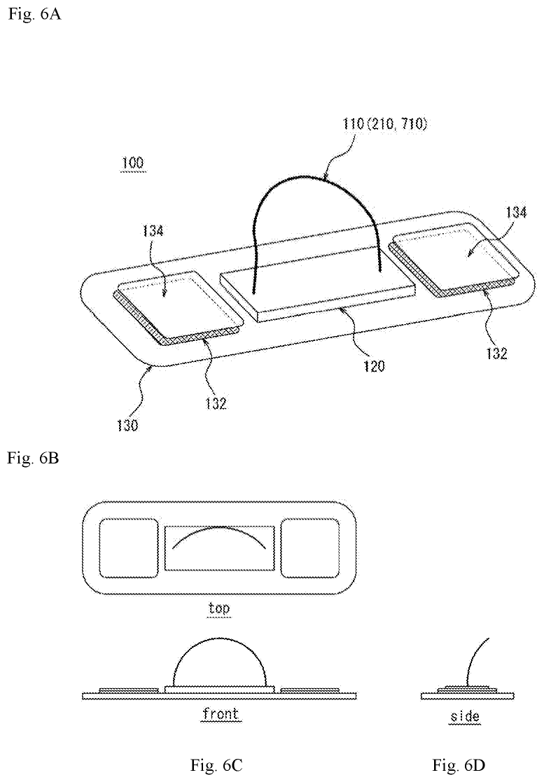

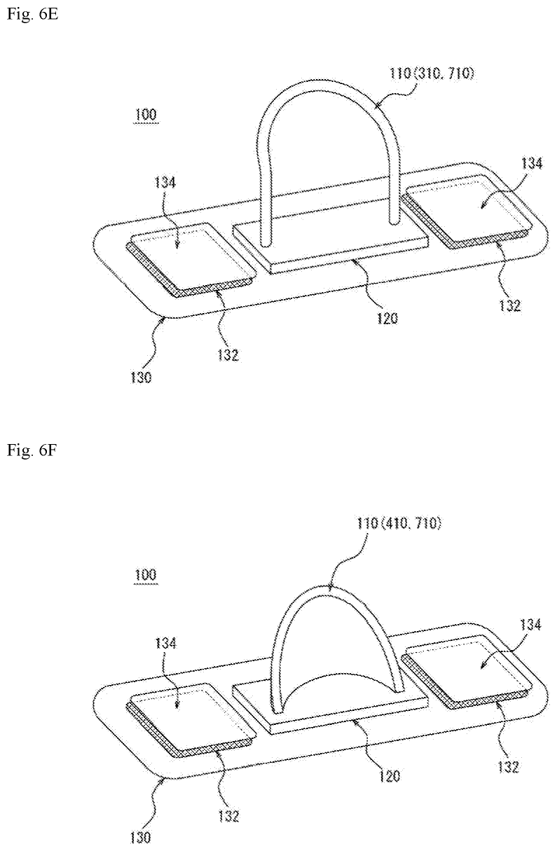

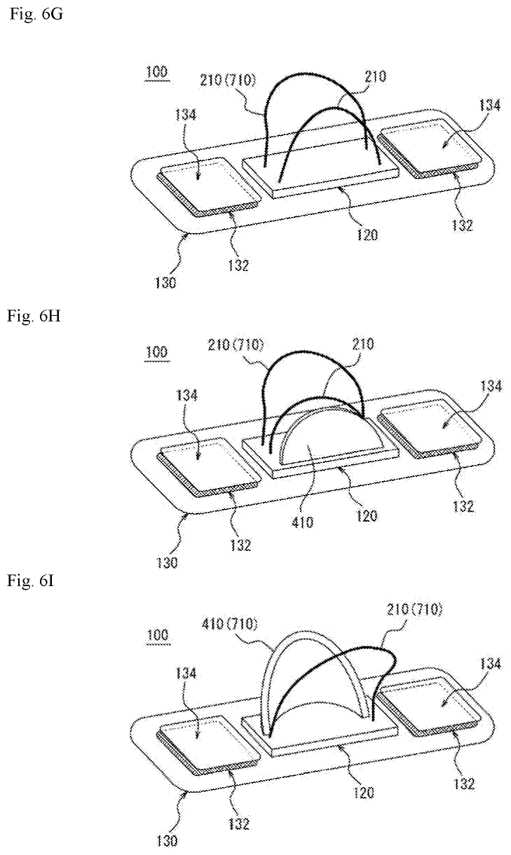

All the above configurations (210, 310, 410, 510, and 610) of the line-compression feature 110 may vary in the edge curvature of the line-compression feature 110 viewed perpendicular to the base 120. FIGS. 6A, 6B, 6C, and 6D show perspective views of a line-compression feature 110 with varying edge curvature 710. As shown in FIGS. 6G-6I, for example, when there is more than one line-compression feature, any combination of above configurations (210, 310, 410, 510, 610, and 710) may be employed.

The height of the base 120 of the line-compression patch assembly 110 can be, for example, approximately 0.5 mm to 15 mm and does not require to have the same thickness throughout. The base 120 may have any features, for example, protrusions, cutouts, holes, and grooves integrated into its structure. The base 120 may be made of, for example, polycarbonate, ABS, polyethylene, polypropylene, polyurethane, PET, acrylic, silicone, PEEK, or combination of the above. The base 120 may be made of transparent material so that the user can see the blood at the application site. The base 120 may be made of flexible material to conform to the patient's skin and movement.

The height of the patch feature 130 of the line-compression patch assembly 110 can be, for example, approximately 0.1 mm to 5 mm and does not require to have the same thickness throughout. The patch feature 130 may have any features, for example, protrusions, cutouts, holes, and grooves integrated into its structure. The patch feature 130 may be made of flexible material to conform to the patient's skin and movement, for example, polyester, polyethylene, polypropylene, polyurethane, PVC, nitryl, silicone, or combination of the above. The patch feature 130 may be made of transparent material so that the user can see the blood at the application site.

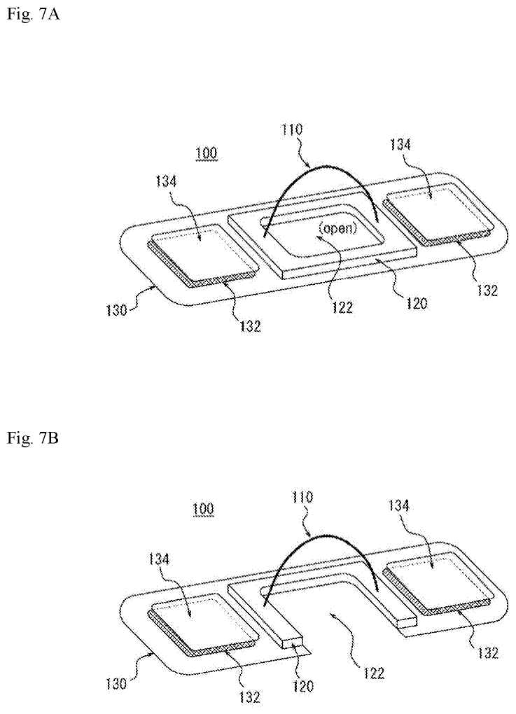

FIGS. 7A and 7B are perspective views of a line-compression patch assembly 100 with the base 120 and the patch feature 130 with a relief feature 122. The relief feature 122 may be an opening or a cutout with the perimeter of the base 120 and the patch feature 130 connected all around (FIG. 7A) or a cutout with one side open (FIG. 7B). The relief feature 122 serves three purposes; the first is to create room for the sheath(s) and/or catheter(s) that are already in the patient to pass through the patch assembly 100, the second is to position the patch assembly 100 in the desired location based on the location of the sheath(s) and/or catheter(s), and the third is to allow the user to directly observe the wound site during the application of the patch assembly 100. The relief feature 122 can be, for example, an opening or a cutout of approximately 100 mm² to 900 mm² in any shape.

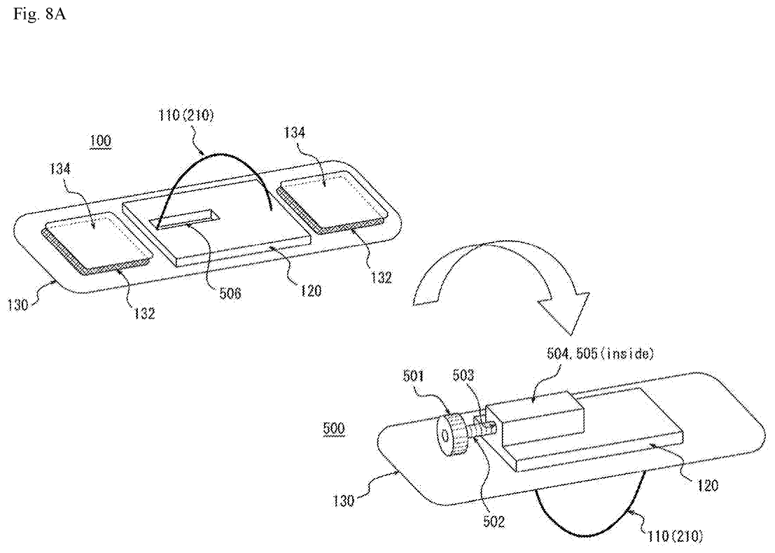

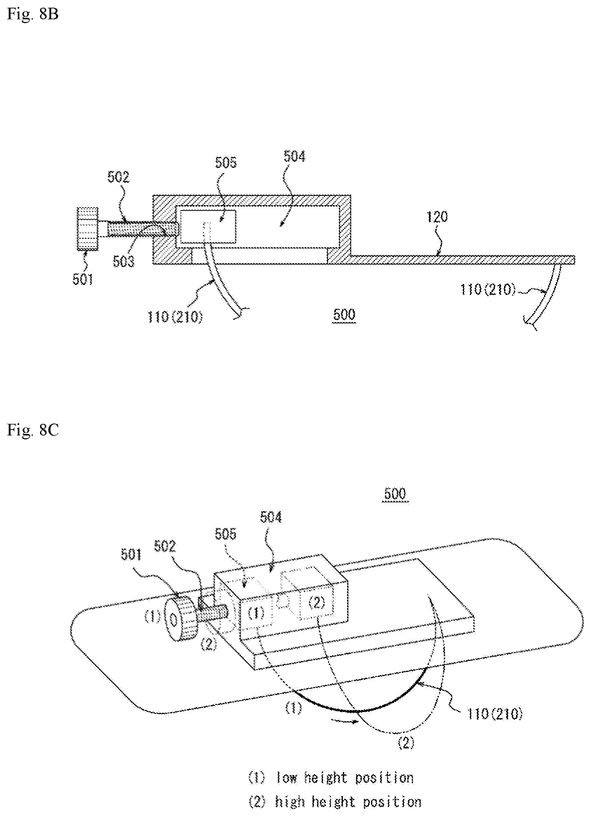

FIGS. 8A, 8B, and 8C are a perspective view, a schematic view, and an illustration of a line-compression patch assembly 100 with an adjustment feature 500, respectively. The adjustment feature 500 is located on the opposite side of the line-compression feature 100 and can be used to adjust the height of the curved wire 210 per patient's anatomy, size and number of the sheaths, and the target area.

As shown in FIGS. 8A and 8B, the adjustment feature 500 consists of the adjustment knob 501, the threaded rod 502, the adjustment rail 504, and the adjustment shuttle 505. The adjustment knob 501 is attached to the end of the threaded rod 502 wherein the other end is housed at the base 120, which has a matching threaded hole 503. The adjustment rail 504 is a feature inside the base 120 on the same end as the threaded hole 503. The adjustment shuttle 505 is a separate block that rests inside the base 120 and can slide along the adjustment rail 504, and to which one end of the curved wire line-compression feature 210 is connected. The other end of the curved wire 210 is attached to the other end of the base 120. The base 120 and the patch feature 130 have a slot 506 to allow the curved wire 210 to change shape when the height is being adjusted.

FIG. 8C is an illustration of the adjustment feature 500 in use to adjust the height of the curved wire 210. When the adjustment knob 501 is turned clockwise, the threaded rod 502 pushes on the adjustment shuttle 505, which moves along the adjustment rail 504 and shortens the base dimension of the curved wire 210, in turn increasing the height of the curved wire 210. When the adjustment knob 501 is turned counterclockwise, the threaded rod 502 retracts back, aided by the spring force of the curved wire 210 pushing on the adjustment shuttle 505, increasing the base dimension of the curved wire 210, in turn reducing the height of the curved wire 210. The thread rod 502 and the thread hole 503 define and lock the adjustment shuttle 505 location, thus defining the height of the curved wire 210.

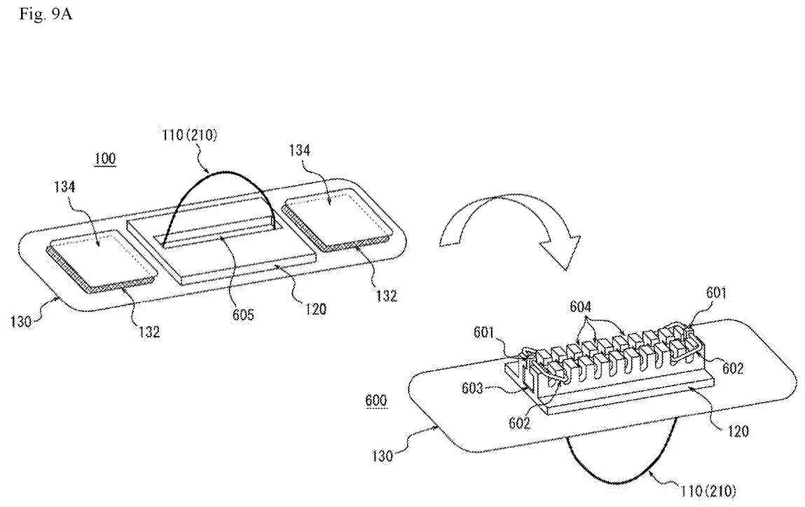

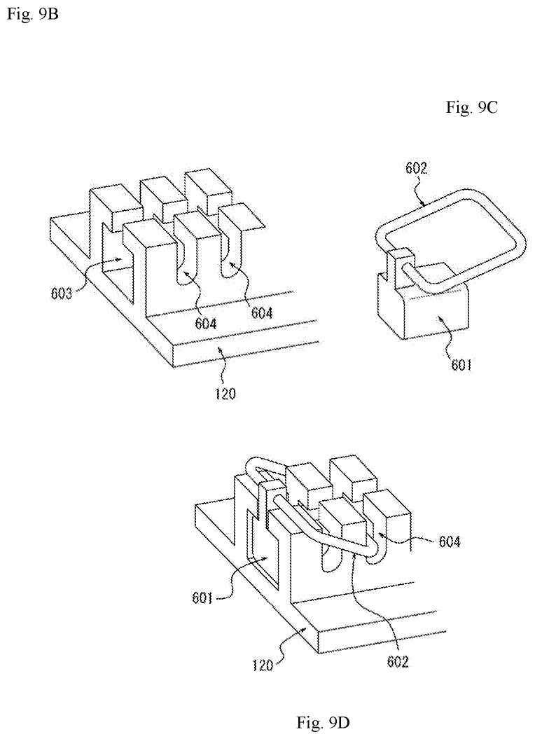

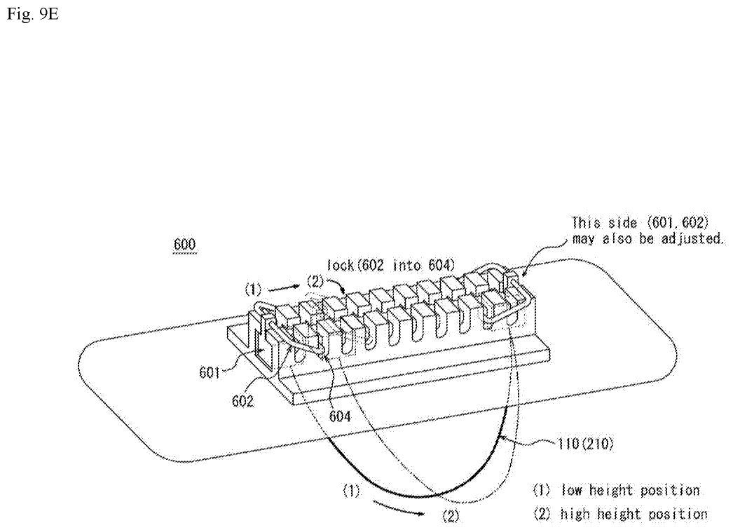

FIGS. 9A, 9B-9D, and 9E are a perspective view, a detailed view, and an illustration, respectively, of a line-compression patch assembly with another adjustment feature 600. The adjustment feature 600 can be used to adjust the height of the curved wire line-compression feature 210 per patient's anatomy, size and number of the sheaths, and the target area.

As shown in FIGS. 9A and 9B, the adjustment feature 600 consists of the adjustment slider(s) 601, the locking feature 602, slider rail 603, and locating groove(s) 604. The adjustment slider 601 is a separate piece that rests and can slide along the slider rail 603, which is a feature within the base 120. In accordance with an exemplary embodiment, there can be two adjustment sliders 601 wherein each end of the curved wire line-compression feature 210 is connected to each slider 601. The locking feature 602 is attached to the adjustment slider 601, which can pivot or flex about the body of the adjustment slider 601. The locating grooves 604 are featured on the base 120 where there may be three to ten such grooves. The base 120 and the patch feature 130 have a slot 605 to allow the curved wire 210 to change shape when the height is being adjusted.

FIG. 9E is an illustration of the adjustment feature 600 in use to adjust the height of the curved wire 210. The locking feature 602 can be engaged in a locating groove 604 by placing the free end into the groove. When the locking feature 602 is engaged with a locating groove 604, the position of the corresponding adjustment slider 601 is defined. Based on the locations of the two adjustment sliders 601, each end of the curved wire 210 position is set, thus also defining the height of the curved wire 210. The height of the curved wire 210 can be adjusted by disengaging the locking feature 602 from the locating groove 604 by pivoting or flexing the locking feature 602, sliding the adjustment slider 601 along the slider rail 603, then engaging the locking feature 602 into another locating groove 604 where the desired height of the curved wire 210 can be achieved.

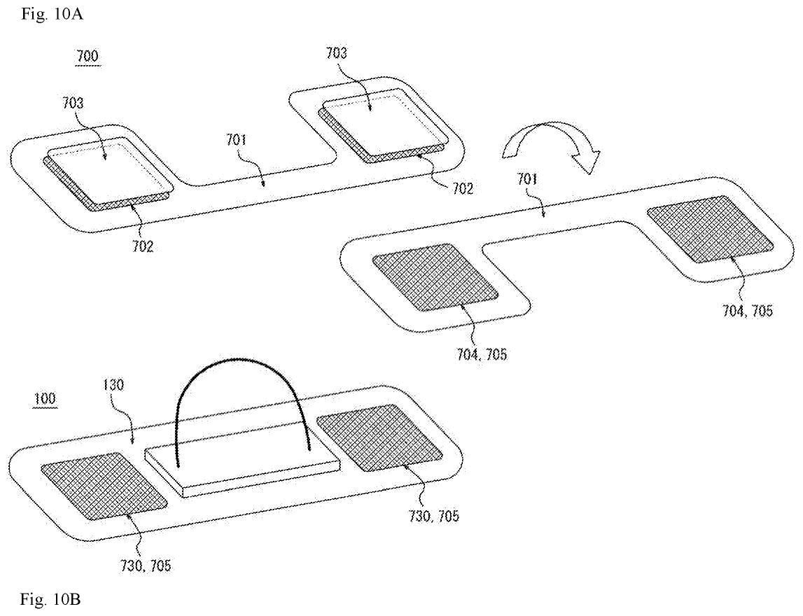

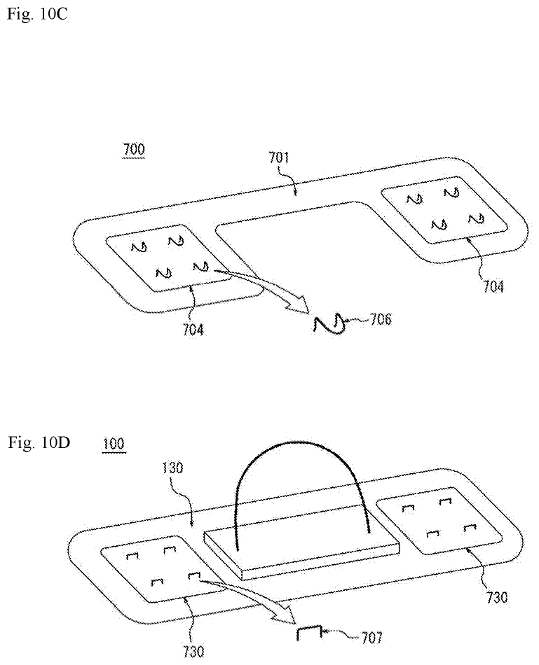

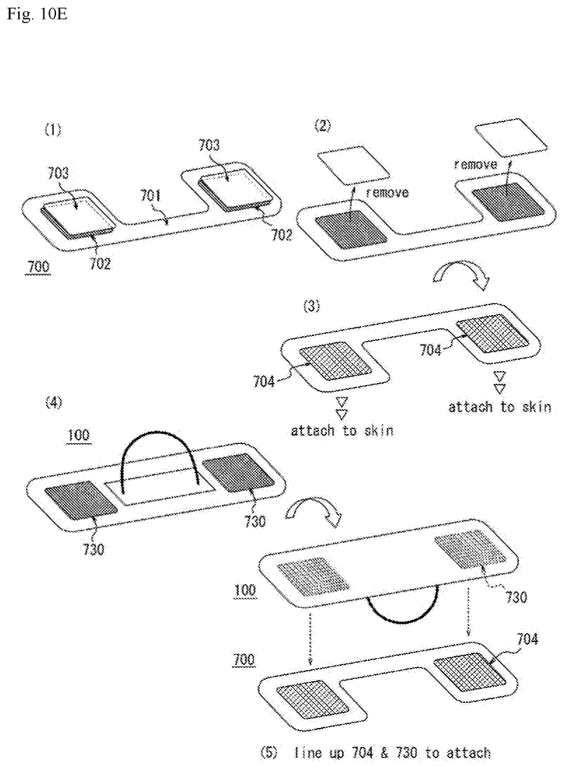

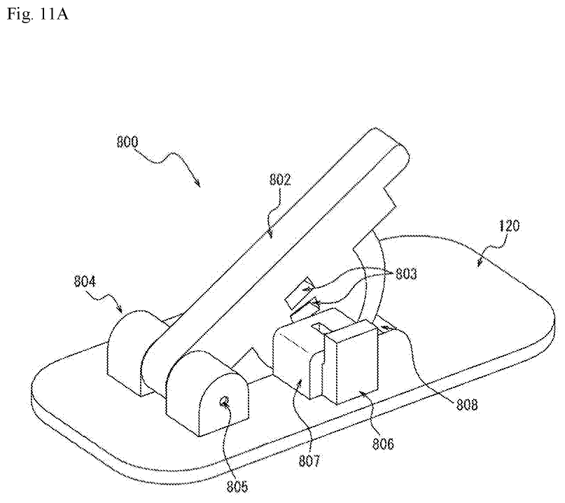

FIGS. 10A and 10B show perspective views of the separate adhesion patch 700 that consists of the patch 701, the skin adhesion feature 702, the skin adhesion cover 703, and the patch assembly adhesion feature 704. The skin adhesion feature 702 is on the surface of the patch 701 that meets the patient's skin, and is covered by the adhesion cover 703 until the separate adhesion patch 700 is ready to be adhered to the patient. On the other side of the separate adhesion patch 700, the patch assembly adhesion feature 704 is attached to the patch 701 wherein the texture is Velcro®-like 705 with its mating secondary adhesion feature 730 is also Velcro®-like 705 on the patch feature 130. The separate adhesion patch 700 may be made of flexible material to conform to the patient's skin and movement, for example, polyester, polyethylene, polypropylene, polyurethane, PVC, nitryl, silicone, or combination of the above. The separate adhesion patch 700 may be made of transparent material so that the user can see the blood at the application site.

FIGS. 10C and 10D shows perspective views of the separate adhesion patch 700 with the patch assembly adhesion feature 704 employing hook-like feature(s) 706 and the mating secondary adhesion feature 730 on the patch feature 130 utilizing loop-like features(s) 707 to connect with the hook-like feature(s) 706. The hook-like feature(s) 706 on the patch assembly adhesion feature 704 and the loop-like feature(s) 707 on the secondary adhesion feature 730 may be reversed; that is, the loop-like feature(s) 707 may be on the surface of the patch assembly adhesion feature 704 and the hook-like feature(s) 706 may be on the mating secondary adhesion feature 730.

FIG. 10E is an illustration of the separate adhesion patch 700 in use. The separate adhesion patch 700 can be placed on the patient first, separately from the line-compression patch assembly 100, and then the secondary adhesion feature 730 on the patch feature 130 can be used to adhere the line-compression patch assembly 100 to the separate adhesion patch 700 already on the patient. In accordance with an exemplary embodiment, this gives the user an ability to adjust or remove the line-compression patch assembly 100 without compromising the adhesive directly on the patient's skin.

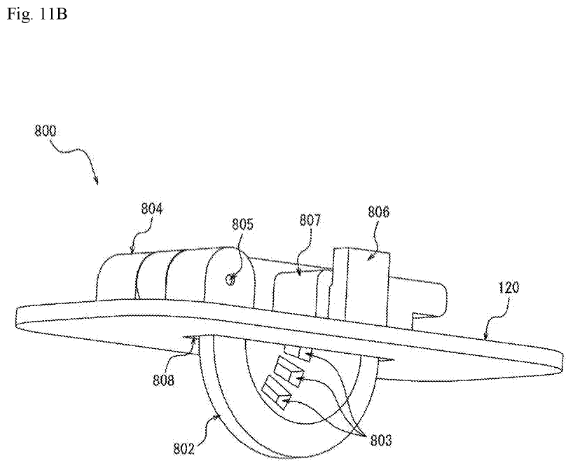

FIGS. 11A and 11B show a line-compression patch assembly 100 with a pivoting adjustment feature 800. The pivoting adjustment feature 800 consists of the pivoting feature 802, the pivot housing 804, the pivot pin 805, the sliding lock 806, and the sliding lock housing 807. The pivot housing 804 and the sliding lock housing 807 are part of or attached to the base 120. The pivoting feature 802 rotates about the pivot pin 805 that rests within the pivot housing 804. The sliding lock 806 is contained by the sliding lock housing 807 wherein the sliding lock 806 can slide back and forth within the sliding lock housing 807 in the direction perpendicular to the pivoting feature 802. The pivoting feature 802 has locking grooves 803 on the side whose function is to provide various locking positions for the sliding lock 806 in setting desired height of the pivoting feature 802. The base 120 and the patch feature 130 have a slot 808 to allow the pivoting feature 802 to move when the height is being adjusted.

The detailed description above describes a line-compression patch configured to achieve hemostasis at femoral vein access site in a living body and a method thereof. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A line-compression patch configured to achieve hemostasis in a living body, the line-compression patch comprising:
a line-compression feature extruding out towards a patient contact surface;
a base providing the support to the line-compression feature; and
a patch feature that provides stability to the line-compression patch by adhering to the patient contact surface.

2. The line-compression patch according to claim 1, wherein the line-compression feature is a curved wire having a peak configured to be in alignment with a patient's vein or wound channel.

3. The line-compression patch according to claim 2, wherein the curved wire is stainless steel, nickel titanium (NiTi) alloy, Elgiloy, polypropylene, polyethylene, or polyethylene terephthalate (PET), having a distance between the curved wire ends on the base of approximately 10 mm to approximately 50 mm, and a distance from the base to the peak of approximately 3 mm to approximately 20 mm.

4. The line-compression patch according to claim 3, further comprising:
a tubular element or a portion of a tubular element having a different texture and material than the curved wire and configured to be placed over the curved wire.

5. The line-compression patch according to claim 1, wherein the line-compression feature is a rectangular shaped-rod.

6. The line-compression patch according to claim 1, wherein the line-compression feature is a plate having a semicircular shape or a rectangular shape.

7. The line-compression patch according to claim 6, wherein the plate having the semicircular shape or the rectangular shape has a uniform thickness.

8. The line-compression patch according to claim 7, wherein the plate having the semicircular shape or the rectangular shape has a tapered thickness, and wherein the tapered thickness comprises a thickness at a base of the plate that is greater than a thickness of the plate an edge of the plate, and wherein the edge of the plate extrudes out towards the patient contact surface.

9. The line-compression patch according to claim 1, wherein the line-compression feature comprises a plurality of curved wires, each of the plurality of curved wires having a peak configured to be in alignment with a patient's vein or wound channel.

10. The line-compression patch according to claim 9, wherein one or more of the plurality of curved wires are arranged or oriented in a direction different from another of the plurality of curved wires, and wherein each of the plurality of curved wires are oriented at an angle between the line compression feature and the base of approximately 30° to approximately 90°.

11. The line-compression patch according to claim 1, wherein the line-compression feature comprises a plurality of curved wires and/or plates, each of the plurality of curved wires and/or plates having a peak and/or edge configured to be in alignment with a patient's vein or wound channel.

12. The line-compression patch according to claim 2, wherein the curved wire has a varying edge curvature.

13. The line-compression patch according to claim 1, wherein the base has an opening or a cutout within a perimeter of the base or an opening or a cutout with an open side, and wherein the opening or the cutout within the perimeter, or the opening or the cutout with the open side are configured to receive at least one sheath and/or at least one catheter, assist with placing the line-compression patch in a desired location, and/or allow a user to directly observe a wound site during an application of line-compression patch.

14. The line-compression patch according to claim 2, further comprising:
an adjustment feature arranged on an opposite side of the line-compression feature and configured to adjust a height of the curved wire.

15. The line-compression patch according to claim 1, wherein the line-compression feature is a pivoting adjustment feature, the pivoting adjustment feature consisting of a pivoting feature, a pivot housing, a pivot pin, a sliding lock, and a sliding lock housing and wherein the pivoting feature rotates about the pivot pin that rests within the pivot housing.

16. The line-compression patch according to claim 1, further comprising:
an adhesion cover, the adhesion cover configured to cover the adhesion feature until the patch feature is ready to be adhered to the patient.

17. The line-compression patch according to claim 1, wherein the patch feature further includes a pair of adhesion features arranged on each side of the line-compression feature.

18. The line-compression patch according to claim 1, wherein the line-compression feature and the base are fixed to the patch feature.

19. The line-compression patch according to claim 1, wherein the line-compression feature and the base are detachable from the patch feature.

20. A line-compression patch configured to achieve hemostasis in a living body, the line-compression patch comprising:
a separate adhesion patch that consists of a patch, a skin adhesion feature, a skin adhesion cover, and a patch assembly adhesion feature, and wherein the skin adhesion feature is on a front side of the patch that meets a patient's skin, and is covered by the skin adhesion cover until the separate adhesion patch is ready to be adhered to the patient, and on a back side of the separate adhesion patch, the patch assembly adhesion feature is attached to the patch; and
an adhesion feature having a line-compression feature extruding out towards a patient contact surface, and wherein the adhesion feature is configured to connect to the patch assembly adhesion feature.

21. A method of achieving hemostasis in a living body, the method comprising:
placing a line-compression patch on a wound channel of the living body, the line-compression patch including a line-compression feature extruding out towards a patient contact surface, a base providing the support to the line-compression feature; and a patch feature that provides stability to the line-compression patch by adhering to the patient contact surface; and
achieving hemostasis with the line-compression patch.

22. The method of claim 21, further comprising:
placing the line-compression patch on the wound channel by pressing the line-compression feature against a target area of a patient and placing the patch feature flat against the patient's skin.

23. The method of claim 21, further comprising:
removing the line-compression patch from the wound after the achieving of hemostasis.

24. The method of claim 21, wherein the wound channel is at a femoral vein access site after cardiac ablation, a left atrial appendage closure, or a mitral valve repair by percutaneous catheter procedure.
